(19) United States
(12) Patent Application Publication
(10) Pub. No.: US 2012/0203215 A1
(43) Pub. Date: Aug. 9, 2012

(54) DEVICE FOR OPHTHALMOLOGICAL LASER SURGERY

(76) Inventors: Peter Riedel, Nurnberg (DE); Christof Donitzky, Eckental (DE)

(21) Appl. No.: 13/499,778

(22) PCT Filed: Oct. 5, 2009
(86) PCT No.: PCT/EP09/07106

§ 371 Date: Apr. 2, 2012

(51) Int. Cl. A61F 9/008

(52) U.S. Cl. 606/5

(57) ABSTRACT

An apparatus for ophthalmic laser surgery includes a contact surface for shaping abutment of an eye to be treated, a first radiation-source for making a treatment laser beam available, optical components for directing the treatment laser beam through the contact surface onto the eye and also a measuring device for measuring at least one corneal thickness dimension or/and positional dimension of the eye bearing against the contact surface, whereby the measuring device makes measured data available that are representative of the measured at least one thickness dimension or/and positional dimension. The measuring device preferably serves for positional surveying of the corneal posterior surface of the eye, whereby an electronic evaluating and control arrangement connected to the measuring device brings about a focus control of the treatment laser beam in a manner depending on the measured position of the posterior surface of the cornea.

This is a United States national phase application of co-pending international application number PCT/EP2009/007106 filed on Oct. 5, 2009, the disclosure of which is incorporated herein by reference.

### BACKGROUND

The invention relates to an apparatus for ophthalmic laser surgery.

Pulsed laser radiation finds application in numerous techniques for treatment of the human eye. In some of these techniques the eye to be treated is pressed against a transparent contact element which, with its contact surface facing towards the eye, constitutes a reference surface which is to enable a precise positioning of the beam focus in the eye in the z-direction. In this connection the ‘z-direction’ means, in conformity with the notation that is customary in the specialist field, the direction of propagation of the laser beam. The plane orthogonal to this direction, on the other hand, is customarily designated as the x-y plane. In particular, treatment techniques that serve for generating incisions in the ocular tissue by means of focused femtosecond laser radiation (the generation of an incision in the human eye by means of pulsed femtosecond laser radiation is always based on the effect of so-called laser-induced optical breakthrough, which results in a photodisruption) frequently make use of such contact elements, in order thereby to define unambiguously the position of the anterior surface of the eye in the coordinate system of the laser apparatus. By the contact element being pressed against the eye in such a way that a closely fitting planar abutment of the eye arises on the contact surface of the contact element facing towards the eye, the contact element presets the z-position of the anterior surface of the eye.

### SUMMARY OF EXAMPLE EMBODIMENTS

The local control of the beam focus in the z-direction is always undertaken with reference to a known reference point or a known reference surface in the coordinate system of the laser apparatus. Depending on the type of treatment, differing reference points or reference surfaces may serve as reference for the z-control of the beam focus.

One form of treatment in which a corneal incision is generated by laser technology is so-called fs LASIK. In this form of treatment a small anterior cover disc of the cornea, designated in the specialist field as a flap, is cut free by means of femtosecond laser radiation. Subsequently, as in the classical LASIK technique (LASIK: Laser In Situ Keratomileusis), the flap which is still attached to the remaining corneal tissue in a hinge region is folded aside, and the tissue exposed in this way is machined in ablating manner by means of UV laser radiation. Another form of treatment is so-called corneal lenticule extraction, in which a small lenticular disc is excised all around within the corneal tissue by means of femtosecond laser radiation. This small disc is subsequently taken away through an additional incision which is guided out to the surface of the eye (the additional incision is produced either by means of a scalpel or likewise by means of femtosecond laser radiation).

In the stated forms of treatment—fs LASIK and corneal lenticule extraction—the guidance of the incision within the eye is undertaken, as a rule, with reference to the contact surface against which the eye is resting. The position of the contact surface within the coordinate system of the laser apparatus is either known or can be easily measured.

There are other forms of treatment in which a referencing of the guidance of the beam to other reference surfaces enters into consideration. One such form of treatment is corneal endothelial keratoplasty, which serves for the treatment of posterior diseases of the cornea. In this connection the diseased rear corneal layer is excised using laser technology and is replaced by a healthy graft. This lamellar technique of posterior keratoplasty is also designated, in a special form, as Descemet's stripping automated endothelial keratoplasty (DSAEK).

For the success of the operation, it is important to be able to cut exactly the endothelial lamella to be removed with the desired thickness. The guidance of the incision is therefore expediently undertaken with reference to the corneal posterior surface. In order to determine the position thereof within the coordinate system of the laser apparatus, the thickness of the cornea, for example, can be measured. With knowledge of the position of the contact surface of the contact element and of the thickness of the cornea (i.e. the z-dimension of the cornea), the position of the corneal posterior surface in the coordinate system of the laser apparatus can be ascertained. With knowledge of the position of the corneal posterior surface, depending on the desired thickness of the lamella the necessary course of the incision within the cornea can then be determined.

Knowledge of the corneal thickness is in many cases necessary or at least desirable. For example, before or even during a laser ablation of the cornea within the scope of a LASIK treatment the thickness of the cornea is measured at least once, but sometimes also repeatedly, for instance in order to be able to ascertain the maximally possible removal of material or to be able to monitor the course of the treatment. In this connection the corneal thickness is always measured in a state in which the eye is not pressed against a contact element and the cornea is accordingly undeformed.

If a thickness value measured in such a state is used in order to ascertain the position of the corneal posterior surface in the coordinate system of the laser apparatus, inaccuracies may arise. For as a consequence of the deformation of the cornea when the eye is pressed against the contact surface the thickness of the cornea measured in the z-direction may change. This applies, in particular, in the case of a levelling of the cornea by an applanation plate with a flat plate underside (the underside in this connection means the side of the applanation plate facing towards the eye). In comparison with the ‘free fall’—that is to say, an undeformed, domed cornea—the measured thickness may differ significantly. The error resulting from this in the ascertainment of the position of the corneal posterior surface has a direct effect on the generated endothelial lamella, the actual thickness of which then under certain circumstances does not correspond to the desired thickness of the incision.

The object of the invention is to make available an apparatus for ophthalmic laser surgery that enables a highly precise placement of corneal incisions.

With a view to achieving this object, in accordance with the invention an apparatus for ophthalmic laser surgery is provided, including a contact surface for shaping abutment of an eye to be treated, a first radiation-source for making a treatment laser beam available, optical components for directing the treatment laser beam through the contact surface onto the eye, and a measuring device for measuring at least one corneal thickness dimension or/and positional dimension of the eye bearing against the contact surface, whereby the measuring device makes measured data available that are representative of the measured at least one thickness dimension or/and positional dimension.

The invention teaches to survey the cornea in the same state of deformation in which the laser treatment also takes place. In this manner, incision deviations can be avoided that may arise if the cornea is surveyed in an undeformed state and the course of the incision and, in particular, the z-control of the beam focus are defined in a manner depending on the measured values in the undeformed state.

The at least one corneal thickness dimension or/and positional dimension may, according to one configuration of the invention, relate to a single point of the cornea in the x-y plane, in particular to a suitably defined point on or at least close to the centre of the cornea. According to another configuration, the at least one thickness dimension or/and positional dimension may relate to various points of the cornea in the x-y plane and may include for each of these points at least one thickness dimension or/and positional dimension. For example, the measuring device may be controlled in such a way that, in accordance with a predetermined pattern of measuring points distributed in the x-y plane, for each of these measuring points it measures at least one corneal thickness dimension or/and positional dimension. Alternatively, the measuring device may be controlled in such a way that it scans at least one predetermined region of the cornea with a plurality of scan points situated closely alongside one another and, for each of these scan points, measures a corneal thickness dimension or/and positional dimension. Such a scanning survey of the cornea permits a high resolution and, so to speak, a planar mapping of the cornea.

The thickness dimension expediently relates to the total thickness of the cornea between its anterior surface and its posterior surface. The positional dimension, on the other hand, relates to the z-position of a predetermined surface of the cornea, in particular its posterior surface.

The measuring device is expediently one that includes a second radiation-source for making a measuring beam available. In this connection the optical components are designed and arranged to direct also the measuring beam through the contact surface onto the eye. This ensures that a survey of the cornea is possible in a state in which the eye is pressed against the contact surface.

The measuring device preferentially includes an optical interferometer which has been set up to cause the measuring beam and a reflected beam coming back from the eye through the contact surface to interfere. For example, the measuring device may be an OLCR measuring device—that is to say, it may operate in accordance with the principle of optical low-coherence reflectometry.

The laser surgical apparatus preferably includes an electronic evaluating and control arrangement connected to the measuring device, which has been set up to bring about a focus control of the treatment laser beam in the direction of propagation of the same (i.e. a z-control of the beam focus) in a manner depending on the measured data. Such a capability of the evaluating and control arrangement is expedient, in particular, for corneal endothelial keratoplasty if the course of the incision for the generation of the endothelial lamella to be removed is defined with reference to the position of the corneal posterior surface in the coordinate system of the laser surgical apparatus. Therefore according to a preferred embodiment the evaluating and control arrangement has been set up to bring about the focus control, dependent on the measured data, of the treatment laser beam in the course of the execution of a control program that serves for generating a lamellar corneal endothelial incision.

A transparent contact element constituting the contact surface may take the form either of an applanation plate or of a contact lens with non-planar abutment surface for the eye. The term ‘applanation plate’ in this connection is understood to mean a contact element that on its plate side facing towards the eye exhibits a flat abutment surface for the front of the eye and therefore permits a levelling of the cornea. On its plate side facing away from the eye the applanation plate may equally be flat; but it may also be concavely or convexly curved there. The term ‘contact lens’, on the other hand, is understood to mean a contact element that on its side facing towards the eye exhibits a non-planar abutment surface for the front of the eye. As a rule, this abutment surface will be concavely curved.

The applanation plate or the contact lens may, for example, be held on a patient adapter which is coupled with a focusing objective of the apparatus.

The pulse duration of the treatment laser beam preferentially lies within the femtosecond range.

According to a further aspect, in accordance with the invention in addition a method is provided for application in the course of the implementation of a corneal endothelial keratoplasty an a human eye. The method includes the following steps:
establishing a shaping abutment contact between the eye and a contact surface,
registering at least one positional dimension of the corneal posterior surface of the eye bearing against the contact surface, and making measured data available that are representative of the registered at least one positional dimension, and
generating control data for the focus control of a treatment laser beam in a manner depending on the generated measured data.

The registration of the positional dimension of the corneal posterior surface may, for example, include a survey of the thickness of the cornea, whereby given knowledge of the position of the contact surface in the coordinate system of the laser surgical apparatus the position of the corneal posterior surface in the coordinate system can be ascertained from this position and from the measured thickness of the cornea. It is similarly possible to measure the position of the corneal posterior surface in the coordinate system of the laser surgical apparatus directly—that is to say, without the intermediate step of the measurement of the corneal thickness and without reference to the position of the contact surface.

The generated control data may, for example, serve for focus control in the course of the generation of a lamellar corneal endothelial incision.

### BRIEF DESCRIPTION OF THE DRAWINGS

The invention will be elucidated further in the following on the basis of the appended drawings. Represented are:

FIG. 1 greatly schematised, an embodiment of an apparatus for ophthalmic laser surgery and

FIG. 2a measuring signal that can be obtained with a measuring device contained in the laser surgical apparatus according to FIG. 1.

### DETAILED DESCRIPTION OF THE DRAWINGS

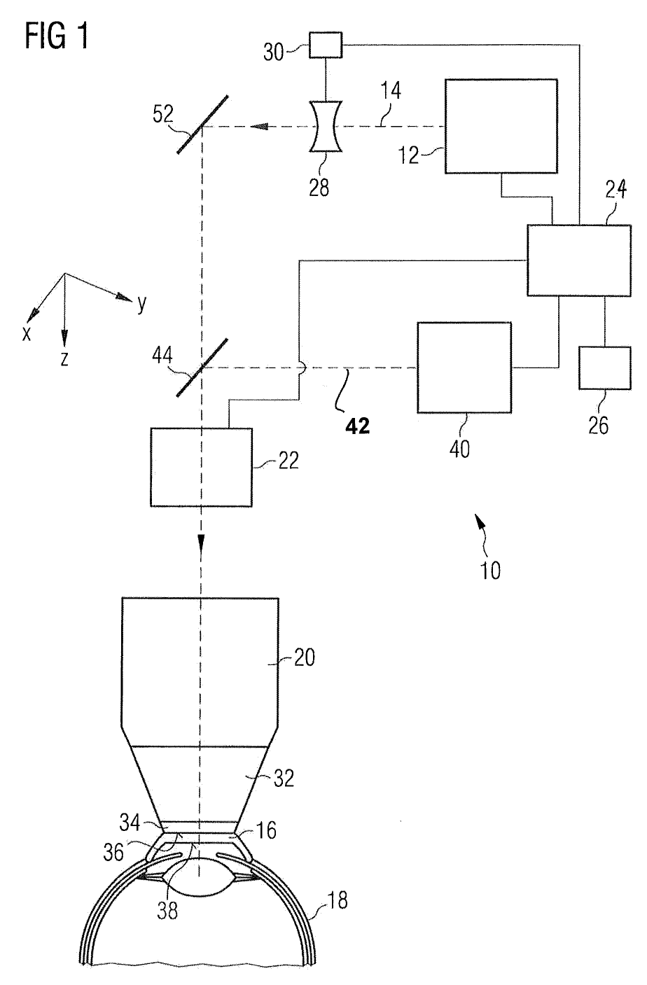

The laser surgical apparatus shown in FIG. 1—denoted generally by 10—exhibits an fs laser 12 which emits a pulsed laser beam 14 with pulse durations within the femtosecond range. The laser beam 14 serves for treating a cornea 16 of a human eye 18. In particular, it serves for generating incisions in the cornea 16, whereby the incision arises as a result of a stringing-together of intracorneal photodisruptions which are brought about in the beam focus through the effect of the laser-induced optical breakthrough.

In the beam path of the laser beam 14 various optical components for guiding and shaping the laser beam 14 are arranged. In particular, these components include a focusing objective 20 (for example, an f-theta objective) as well as a scanner 22 placed upstream of the objective 20, by means of which the laser beam 14 emitted by the laser 12 is capable of being deflected in a plane (x-y plane) orthogonal to the beam path of the laser beam in accordance with a treatment profile ascertained for the eye 18. A coordinate system which has been drawn in illustrates this plane and also a z-axis predetermined by the direction of the laser beam 14. The scanner 22 is, for example, constructed in a manner known as such from a pair of galvanometrically controlled deflecting mirrors which are respectively responsible for the deflection of the beam in the direction of one of the axes spanning the x-y plane. An electronic evaluating and control unit 24 controls the scanner 22 in accordance with a control program stored in a memory 26, which implements an incision profile to be generated in the eye 18 (represented by a three-dimensional pattern of scan points at which, in each instance, a photodisruption is to be brought about).

Moreover, the aforementioned optical components include at least one controllable optical element 28 for the z-adjustment of the beam focus of the laser beam 14. In the case that is shown, this optical element 28 is constituted by a lens (in concrete terms, a diverging lens). For the purpose of controlling the lens 28, use is made of a suitable actuator 30 which in turn is controlled by the evaluating and control unit 24. For example, the lens 28 may be capable of being mechanically displaced along the beam path of the laser beam 14. Alternatively, it is conceivable to use a controllable liquid lens of variable refractive power. With z-position unchanged and also with otherwise unchanged setting of the focusing objective 20, a z-relocation of the beam focus can be obtained by displacing a longitudinally adjustable lens or by varying the refractive power of a liquid lens. It will be understood that for the z-adjustment of the beam focus other components are also conceivable, for instance a deformable mirror. On account of its comparatively higher inertia, with the focusing objective 20 it is expedient to perform only an initial basic setting of the beam focus (i.e. focusing onto a predetermined z-reference position), and to bring about the z-relocations of the beam focus predetermined by the incision profile by means of a component with quicker speed of response which is arranged outside the focusing objective 20. Such a component with quicker speed of response is, for example, the lens 28.

On the side of emergence of the beam the focusing objective 20 is coupled with a patient adapter 32 which serves for establishing a mechanical coupling between the eye 18 and the focusing objective 20. Customarily in the case of treatments of the type being considered here a suction ring which is not represented in any detail in the drawing but which is known in itself is mounted onto the eye and fixed there by suction force. The suction ring and the patient adapter 32 constitute a defined mechanical interface which permits a coupling of the patient adapter 32 to the suction ring. In this regard, reference may be made, for example, to international patent application PCT/EP 2008/006962, the total content of which is hereby incorporated by reference.

The patient adapter 32 serves as carrier for a transparent contact element 34 which, in the case shown, takes the form of a plane-parallel applanation plate. The patient adapter 32 includes, for example, a taper-sleeve body, at the narrower end of which (in the drawing, the lower end) the applanation plate 34 is arranged. In the region of the wider end of the sleeve (in the drawing, the upper end), on the other hand, the patient adapter 32 is attached to the focusing objective 20 and possesses there suitable structures which permit a, where desired, releasable fixing of the patient adapter 32 to the focusing objective 20.

Because it comes into contact with the eye 18 during the treatment, the applanation plate 34 is, from the standpoint of hygiene, a critical article which is therefore expediently to be exchanged after each treatment. For this purpose, the applanation plate 34 may have been exchangeably fitted to the patient adapter 32. Alternatively, the patient adapter 32 together with the applanation plate 34 may constitute a disposable unit or at least a unit that is intended for once-only use and then to be sterilised again for further use. In this case the applanation plate 34 may have been permanently connected to the patient adapter 32.

In any case, the underside of the applanation plate 34 facing towards the eye constitutes a flat contact surface 36, against which the eye 18 has to be pressed. This brings about a levelling of the anterior surface of the eye (generally, a deformation of the cornea 16 of the eye 18). The levelling of the anterior surface of the eye (synonymous with the anterior surface of the cornea) also brings about a corresponding orientation of the corneal posterior surface denoted by 38. Because the cornea 16 does not have to have exactly the same thickness everywhere, the posterior surface 38 of the leveled cornea 16 does not necessarily lie exactly parallel to the contact surface 36.

In the case of lamellar corneal endothelial keratoplasty, from the rear region of the cornea 16 a small disc (a so-called lamella) is separated out which is removed and replaced by a healthy lamella. The excision of the posterior corneal lamella is undertaken by means of the laser beam 14. The course of the incision within the cornea is determined in this case by the desired thickness of the lamella. This thickness is measured from the corneal posterior surface 38, which is why it is necessary to know the position of the corneal posterior surface 38 in the coordinate system of the laser surgical apparatus 10, in order that the beam focus of the laser beam 14 can be locationally controlled in such a way that a corneal lamella with the desired thickness in fact arises.

For the purpose of surveying the position of the corneal posterior surface 38, the laser surgical apparatus 10 exhibits an optical-coherence interferometric measuring device 40 which is preferentially an OLCR measuring device. The measuring device 40 emits a measuring beam 42 which by means of an immovably arranged, semi-transparent deflecting mirror 44 is coupled into the beam path of the laser beam 14. The measuring beam 42 passes through the focusing objective 20, the patient adapter 32 and also the applanation plate 34 and impinges on the eye 18. The incidence of the measuring beam 42 on the eye brings about a reflection. The latter finds its way back to the measuring device 40 on the same path that the measuring beam 42 has taken. In an interferometer contained in the measuring device 40 and not represented in any detail, the measuring beam 42 is caused to interfere with the reflected beam coming back. From the measured interference data obtained in this regard, the z-position of the corneal posterior surface 38 in the coordinate system of the laser surgical apparatus 10 can be ascertained. The evaluating and control unit 24 receives the measured interference data from the measuring device 40 and computes from these data the z-position of that point on the corneal posterior surface 38 at which the measuring beam 42 impinged. In the course of the following laser treatment of the eye 18 the evaluating and control unit 24 takes the z-position of the corneal posterior surface 38, ascertained in this way, into account in connection with the z-control of the beam focus, specifically in such a way that the incision is in fact generated at the intended position deep within the cornea 16. For this purpose, the evaluating and control unit 24 references the z-position of the beam focus to be set to the measured z-position of the corneal posterior surface 38.

In the case that is shown, the measuring beam 42 emitted by the measuring device 40 passes through the scanner 22. This makes it possible to utilise the x-y scan function of the scanner 22 also for the measuring beam 42. In this way a scanning of the corneal posterior surface 38 by the measuring beam 42 at different points along the x-y plane is possible. The corneal posterior surface 38 will in its leveled region usually not lie exactly parallel to the x-y plane. A varying thickness of the cornea and also a possible angular position of the contact surface 36 relative to the x-y plane may have the result that the z-position of the corneal posterior surface 38 is different at different points along the x-y plane. In order to take such variations into account, it is advisable to measure the z-position of the corneal posterior surface 38 at various points on the same. In this connection it may be sufficient to perform the measurement only at a limited number of representative measuring points. For example, the surveying of the corneal posterior surface 38 can be carried out in accordance with a pattern that provides a central measuring point as well as further measuring points that are distributed around the central measuring point in one or more concentric circles. The control of the location of the measuring beam in the x-y plane that is necessary for this can expediently be obtained with the scanner 22.

For the regions of the corneal posterior surface 38 situated between the measuring points and not surveyed, the position of the corneal posterior surface 38 in the x-y-z coordinate system can, for example, be modelled or estimated by interpolation or extrapolation.

In one configuration the scanner 22 may contain a pair of mirrors or a deflecting unit operating in accordance with a different scanning technique, which is utilised jointly for the x-y deflection of the laser beam 14 and of the measuring beam 42. In another configuration the scanner 22 may contain separate pairs of mirrors or generally separate deflecting units, one of which is used for x-y deflection of the laser beam 14 and the other for x-y deflection of the measuring beam 42. The deflecting unit for the measuring beam 42 could, for example, be equipped with smaller, more rapidly movable mirrors than the deflecting unit for the laser beam 14. In yet another configuration, a deflecting unit for the laser beam 42 may have been arranged in that part of the beam path of the measuring beam 42 which lies upstream of the deflecting mirror 44.

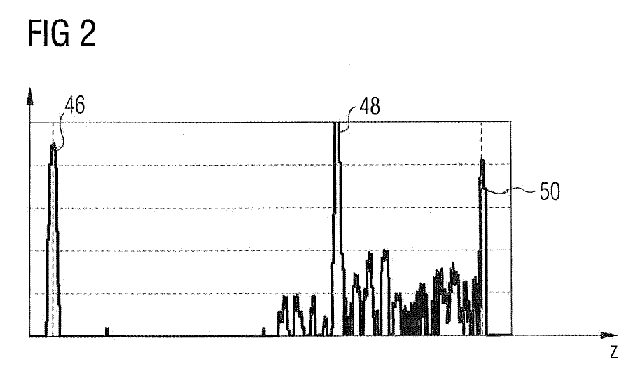

FIG. 2 shows a signal form of a measuring signal that can be obtained from the measuring device 40 at one of the measuring points. In this measuring signal three particularly clearly outstanding signal peaks 46, 48, 50 can be discerned. The left-hand signal peak 46 arises through reflection of the measuring beam 42 on the front of the applanation plate 34 facing away from the eye; the middle signal peak 48 arises through reflection of the measuring beam 42 on the contact surface 36; and the right-hand signal peak 60 is to be attributed to a reflection of the measuring beam 42 on the corneal posterior surface 38. The position of the signal peaks 46, 48, 50 along the abscissa of the axial diagram drawn in FIG. 2 is representative of the position of the surface in question (front of the applanation plate 34, contact surface 36, corneal posterior surface 38) in the z-direction in the coordinate system of the laser surgical apparatus 10. Therefore the abscissa in FIG. 2 is also designated as the z-axis. The reciprocal spacing of the signal peaks 46, 48, 50 along the z-axis in FIG. 2 is accordingly representative of the reciprocal z-spacing of the front of the applanation plate 34, of the contact surface 36 and of the corneal posterior surface 38.

Denoted by reference symbol 52 is a further immovable deflecting mirror which serves for guiding the treatment laser beam 14.

1. Apparatus for ophthalmic laser surgery, including
a contact surface for shaping abutment of an eye to be treated,
a first radiation-source for making a treatment laser beam available,
optical components for directing the treatment laser beam through the contact surface onto the eye,
a measuring device for measuring at least one corneal thickness dimension or/and positional dimension of the eye bearing against the contact surface, whereby the measuring device makes measured data available that are representative of the measured at least one thickness dimension or/and positional dimension.

2. Apparatus according to claim 1, wherein the measuring device includes a second radiation-source making a measuring beam available, and the optical components are designed and arranged to direct also the measuring beam through the contact surface onto the eye.

3. Apparatus according to claim 1, wherein the measuring device is designed to measure, for various points of the cornea, in each instance at least one corneal thickness dimension or/and positional dimension.

4. Apparatus according to claim 1, further including an electronic evaluating and control arrangement connected to the measuring device, which has been set up to bring about a focus control of the treatment laser beam in the direction of propagation of the same in a manner depending on the measured data.

5. Apparatus according to claim 4, wherein the evaluating and control arrangement has been set up to ascertain from the measured data the position of the corneal posterior surface relative to the direction of propagation of the treatment laser beam for at least one point of the cornea and to bring about a focus control of the treatment laser beam, in particular a control of the beam focus in the direction of propagation of the treatment laser beam, in a manner depending on the ascertained position of the corneal posterior surface.

6. Apparatus according to claim 4, wherein the evaluating and control arrangement has been set up to bring about the focus control, which is dependent on the measured data, of the treatment laser beam in the course of the execution of a control program that serves for generating a lamellar corneal endothelial incision.

7. Apparatus according to claim 2, wherein the measuring device includes an optical interferometer which has been set up to cause the measuring beam and a reflected beam coming back from the eye through the contact surface to interfere.

8. Apparatus according to claim 2, wherein the measuring device operates in accordance with the principle of optical low-coherence reflectometry.

9. Apparatus according to claim 1, wherein the contact surface is constituted by a transparent contact element which takes the form of an applanation plate or of a contact lens with non-planar abutment surface for the eye.

10. Apparatus according to claim 9, wherein the applanation plate or the contact lens is held on a patient adapter which is coupled with a focusing objective of the apparatus.

11. Apparatus according to claim 1, wherein the pulse duration of the treatment laser beam lies within the femtosecond range.

12. Method for application in the course of the implementation of a corneal endothelial keratoplasty on a human eye, including the following steps:
establishing a shaping abutment contact between the eye and a contact surface,
registering at least one positional dimension of the corneal posterior surface of the eye bearing against the contact surface, and making measured data available that are representative of the registered at least one positional dimension,
generating control data for the focus control of a treatment laser beam in a manner depending on the generated measured data.

13. Method according to claim 12, wherein the generated control data serve for focus control in the course of the generation of a lamellar corneal endothelial incision.
